(19) United States
(12) Patent Application Publication
(10) Pub. No.: US 2015/0316542 A1
(43) Pub. Date: Nov. 5, 2015

(54) Reference and Normalisation Method for Use With Bead-Based Immunoassays in a Microfluidic Disc

(71) Applicant: RADISENS DIAGNOSTICS LIMITED, Cork (IE)

(72) Inventor: Damian Curtin, Co. Kerry (IE)

(21) Appl. No.: 14/649,646

(22) PCT Filed: Dec. 5, 2013
(86) PCT No.: PCT/EP2013/075729

§ 371 Date: Jun. 4, 2015

Related U.S. Application Data
(60) Provisional application No. 61/733,848, filed on Dec. 5, 2012.

(30) Foreign Application Priority Data
Dec. 5, 2012 (EP) 12195765.8

(51) Int. Cl. G01N 33/542; G01N 21/07; G01N 21/64

(52) U.S. Cl. CPC G01N 33/542; G01N 21/6428; G01N 21/07; G01N 2201/02

(57) ABSTRACT

The invention relates to a microfluidic system and method of processing biological samples in microfluidic system comprising the steps of the steps of positioning at least one detection chamber adapted for receiving particles, said particles comprising a reference label and reporter label, wherein the labels exhibit different wavelength properties when irradiated with light; and normalising the particle reporter label by using any detected magnitude in the measured properties of the reference label.

### FIELD

The invention relates to a reference and normalisation method for use with Bead-Based Immunoassays in a Microfluidic Disc. In particular the invention relates to a reference and normalisation technique related to a microfluidic disc, apparatus, system and method, for use in sandwich immunoassay diagnostic binding reactions which are not concentration limited.

### BACKGROUND

Manual processing to determine the cellular/biological content of various types of samples, and in particular samples that contain living cells, is cost-prohibitive in many applications and is also prone to errors. Automation is also cost-prohibitive in many applications, and is inappropriate as currently practiced—using, for example, liquid handling robots—for applications such as point-of-care or doctor's office analysis. As a result, there is an unmet need to provide sample processing for multiplexed biological assays that is less expensive and less prone to error than current automation or manual processing, such as various point-of-care diagnostic assay systems.

A drawback of point-of-care diagnostic assay systems is that they are typically incapable of multiplexing a variety of assay types. While these systems are quite good at performing a variety of similar assay types—such as lateral flow assays, or electrochemical assays, etc.—the assay conditions required of different kinds of assays—such as immunoassay vs. colorimetric blood chemistry—make them inappropriate for multiplexing these different assay types. Again, centralized laboratories may achieve such integration by splitting samples and performing the assays in different devices. The centrifugal microfluidic platform with optical detection allows for a variety of assay technologies to be implemented in parallel using a single instrument and disposable.

Another significant problem of currently implemented immunofluorescent bead assays is that they are run on instruments developed for hematologic flow cytometry, which are both expensive and complex, requiring significant maintenance and calibration. There are a number of examples in the art which promise cheaper and less complex solutions for point-of-care implementation based on centrifugal microfluidic technology,

One such example in the art is US patent publication number US2004/096867, Gyros Patent AB, which describes a system with trapped beads that are “washed through”, one weakness with this US patent publication is that it does not provide a means for moving beads from one point to another, making single bead detection impossible. A further weakness is that systems with trapped beads forming the solid phase require manufacturing techniques that are demanding.

PCT Patent Publication number WO2006/110098, Gyros Patent AB, discloses a centrifugal based microfluidic device that comprises a microchannel structure in which there is a detection microcavity which in the upstream direction is attached to an inlet microconduit for transport of liquid (transport microconduit) to the detection microcavity and which is used for detecting the result of a reaction taking place in the detection microcavity or in a reaction microcavity positioned upstream of the detection microcavity. This application is primarily directed toward providing means for generating fluid “plug” flow and for joining fluids without bubbles or blockages and requires hydrophobic surface treatment for valves.

In WO9853311A2, Gamera Bioscience Corp, devices are disclosed for the performance of competitive immunoassays on a microfluidic disc. These are performed using a stationary solid phase—e.g., antibodies dried within a chamber on the disc. Furthermore, detection is via color-formation by a substrate specific for binding. While this detection method is attractive in that it allows amplification of signal through the enzymatic reaction causing the build-up of color, but it is far more sensitive to temperature variations than is a simple fluorescent-binding assay. This temperature-sensitivity also makes storage of enzyme-based reagents for long periods (long shelf life) difficult.

In US20040089616, Kellogg et al. discloses a microfluidic disc for evaluation of glycated haemoglobin, total haemoglobin, and glucose in whole blood. One portion of the microfluidic disc uses an affinity matrix comprised of agarose beads retained between frits within the flow path of lysed, dilute blood: The glycated fraction is bound to the beads as it flows through, and the non-glycated fraction is measured photometrically in a cuvette. Combined with the measurement of total haemoglobin in another cuvette, this provides the glycated haemoglobin fraction. There are several problems to this formatting of an affinity method. First, retention of the beads requires either a) inserted elements, such as frits or b) channel constrictions in 1 dimension (“weirs”, which are too shallow for the passage of a bead) or 2 dimensions to retain the solid phase beads. In each case, manufacturing requirements are significant, requiring specialized methods for the production of features as low as a few to a few tens of microns.

In US005822071A, Dosmann and Nelson discloses a system for normalising measurements obtained from spectrometers to correct for measurement biases in individual spectrometers. A normalisation factor is obtained by placing a holographic dispersion filter with known characteristics in the spectrophotometer and comparing to the actual measured value. Future measured results are then normalised by multiplying by the stored normalisation factor. The approach described uses a stationary cuvette based system.

Other patent publications include US2012/0291538, assigned to 3M Innovative Properties Company Limited, discloses a system for volumetric metering on a sample processing device. However, in sandwich immunoassay binding reactions which are not concentration limited—that is, in which neither analyte nor reagent depletion is significant—the degree of analyte binding to the bead's surface in a prescribed tie is primarily due to the analyte concentration. For each bead, a larger number of analyte molecules directly map to a larger number of fluorescent labels which are detected. This will be observed in a flow channel detection system. Similarly, if all beads are pelleted and detected, the integrated fluorescence directly corresponds to the amount of analyte bound, which is a function of both the concentration (determining individual bead fluorescence) and the total number of beads. In some cases when beads are transported from point to point on the device, the precise number of beads—and thus number of binding molecules—cannot be fixed. A problem is that beads can be lost due to binding to an internal surface of the disc.

Other problems include errors associated with light excitation and collection variation from various physical non-idealities such as positioning variation, vibration, disk surface abnormalities and variance in bead packing or depth of field, may introduce much imprecision in the immunoassay measurement process.

It is therefore an object of the invention to provide an effective system and method to analyse sandwich immunoassay binding reactions which are not concentration limited to overcome at least one of the above mentioned problems.

### SUMMARY OF THE INVENTION

According to the invention there is provided, as set out in the appended claims, a microfluidic system for processing biological samples comprising:
a platform coupled to a rotary motor and adapted to provide at least one detection chamber for receiving particles, said particles comprising a reference label and reporter label, wherein the labels exhibit different wavelength properties when irradiated with light; and
means for normalising the particle reporter label in the detection chamber by using any detected variance in the measured properties of the reference label.

The invention provides a bead-based sandwich immunoassay method on a centrifugal microfluidic platform, using reporter fluorescent labelling methods known to those skilled in the art, but where a second reference label is used as a normalising reference.

With this approach beads with a reference label, constant across all beads used within the immunoassay, are used. This reference label fluoresces at a different wavelength to the reporter label. As the reference label is constant across each bead, any variance in the measured fluorescence of the reference label is due to physical non-idealities.

As these non-idealities identically act upon the reporter label, then the variance of the measured fluorescence of the reference label can be used as a correction or normalising factor for the measured fluorescence of the reporter label, thus minimising the measured imprecision of the immunoassay binding reactions.

In one embodiment the means for normalising comprises determining the magnitude of the measured fluorescence of the reference label, wherein the determined magnitude provides a correction factor for the measured fluorescence of the reporter label.

In one embodiment the measured properties of the reference label comprises measured fluorescence properties of the reference label.

In one embodiment the measured fluorescence properties of the reference numeral provides a normalising factor for measured fluorescence of the reporter label.

In one embodiment the normalising factor is obtained by dividing the reporter label fluorescence measurement by the reference label fluorescence measurement, such that a normalised immunoassay signal will be corrected for any errors in the system.

In one embodiment the particles are immuno-modified beads and/or fluorescently labelled immuno-modified beads representative of characteristics of said biological sample.

In one embodiment the detection zone is adapted to cooperate with an optical system while the platform rotates.

In one embodiment the particle receiving structure comprises a pelleting chamber.

In one embodiment the particle receiving structure comprises a flow channel.

In a further embodiment of the invention there is provided a method of processing biological samples in a microfluidic system comprising the steps of:
positioning at least one detection chamber adapted for receiving particles, said particles comprising a reference label and reporter label, wherein the labels exhibit different wavelength properties when irradiated with light; and
normalising the particle reporter label by using any detected variance in the measured properties of the reference label.

In one embodiment the method comprises the step measuring properties of the reference label by measuring fluorescence properties of the reference label.

In one embodiment the measured fluorescence properties of the reference numeral provides a normalising factor for measured fluorescence of the reporter label.

In one embodiment the method comprises the step of obtaining the normalising factor by dividing the reporter label fluorescence measurement by the reference label fluorescence measurement, such that a normalised immunoassay signal will be corrected for any errors in the system.

In one embodiment the particles are immuno-modified beads and/or fluorescently labelled immuno-modified beads representative of characteristics of said biological sample.

In one embodiment the particle receiving structure comprises a pelleting chamber.

In a further embodiment there is provided a microfluidic system for processing biological samples comprising:
a platform coupled to a rotary motor and adapted to provide at least one detection chamber for receiving particles, said particles comprising a reference label and reporter label, wherein the labels exhibit different wavelength properties when irradiated with light; and
a module configured for normalising the particle reporter label in the detection chamber by using any detected variance in the measured properties of the reference label.

In another embodiment there is provided a method to analyse sandwich immunoassay binding reactions which are not concentration limited in a microfluidic system comprising the steps of:
positioning at least one detection chamber adapted for receiving particles, said particles comprising a reference label and reporter label, wherein the labels exhibit different wavelength properties when irradiated with light; and
normalising the particle reporter label by using any detected variance in the measured properties of the reference label.

Use of a reference label from a particle to establish if an adequate number of particles are present in a detection chamber for a viable immunoassay measurement according to the method as hereinbefore described.

In one embodiment the signal level of the reference label can also be used to infer whether sufficient bead numbers are being detected in a bead pellet.

There is also provided a computer program comprising program instructions for causing a computer program to carry out the above method which may be embodied on a record medium, carrier signal or read-only memory.

### BRIEF DESCRIPTION OF THE DRAWINGS

The invention will be more clearly understood from the following description of an embodiment thereof, given by way of example only, with reference to the accompanying drawings, in which:

FIG. 1 illustrates a bead-based sandwich immunoassay construct, using a reporter label and reference label according to a preferred embodiment of the invention;

FIG. 2 illustrates an alternative detection scheme in which beads are sedimented into a volume and then detected according to a preferred embodiment of the invention; and

FIG. 3 illustrates schematically a disc structure which provides a dilution or wash process followed by injection of beads into a flow channel.

### DETAILED DESCRIPTION OF THE INVENTION

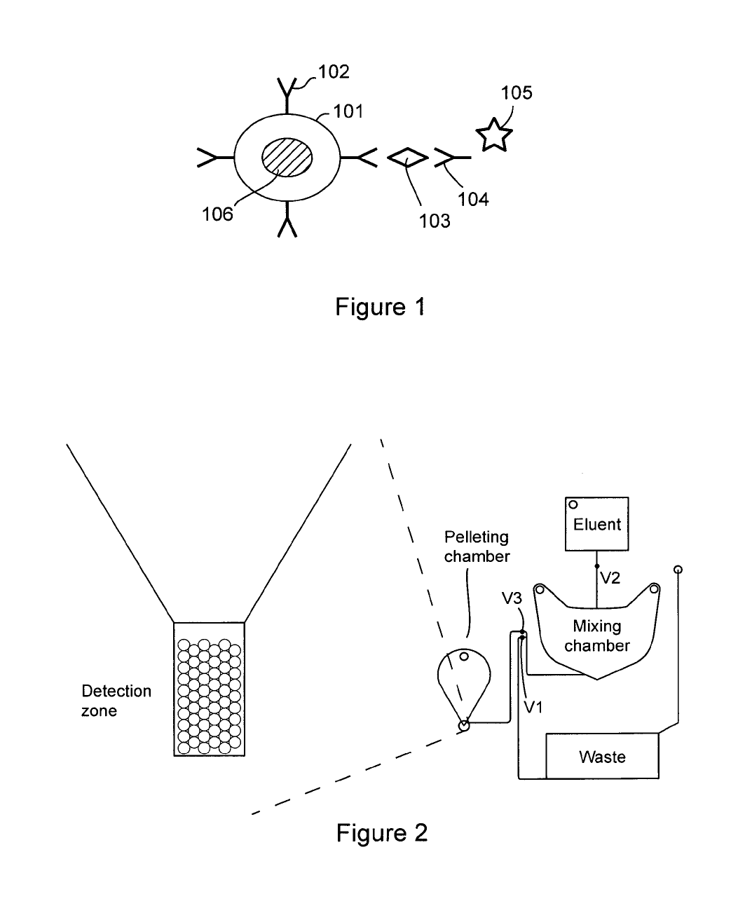

FIG. 1 illustrates a bead-based sandwich immunoassay construction comprising a bead 101 with capture antibodies 102 conjugated to the beads surface. These antibodies capture the target analyte 103, which in turn are conjugated with a detection antibody 104. These detection antibodies are labelled with a fluorescent label 105, which when irradiated with an incident light source emit at a wavelength as a Stokes shift from the incident wavelength. The present invention uses a bead with an embedded reference label 106 that remains constant across the bead set used in the immunoassay. The excitation wavelengths of the reporter and reference labels maybe similar, whereas their respective emission wavelengths differ.

FIG. 2 illustrates schematically a disc structure which provides various upstream sample processing steps, as described in the art, followed by centrifugation of the beads into a bulk pelleting chamber for subsequent detection. Such a disc structure is described in PCT patent application number PCT/IE2012/000026 assigned to Radisens Diagnostics Limited and incorporated herein by reference. It is understood that other disc structures not shown can be used for preparation of the sample (e.g., plasma separation) and delivery of liquid reagents and sample to the mixing chamber which is shown. The beads are transported to a second pelleting chamber or particle receiving structure. The pelleting chamber is shaped such that there is a small, shallow detection zone at its outermost point. In this way fluorescent beads are compacted into a small area upon pelleting by centrifugation that may be interrogated in its entirety by the optical system.

The compound label fluorescence from the plurality of bead sandwiches in the pelleting chamber is measured either statically or as the disc rotates over the pelleting chamber using a first optical detection channel known to those skilled in the art. Simultaneously, the reference label fluorescence from the beads in the pelleting chamber is similarly measured by a second optical detection channel. Upon Mathematical adjustment of the reporter label fluorescence measurement by the reference label fluorescence measurement, the resulting normalised immunoassay signal will be corrected for the various non-idealities previously mentioned.

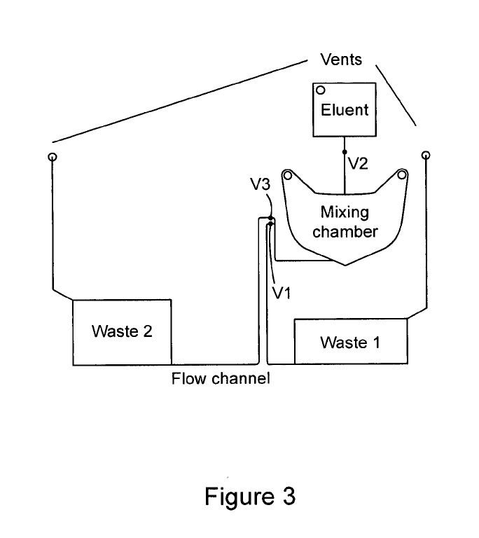

In another embodiment a detection mechanism that can utilise the invention beyond the described pelleted bulk detection is a flow channel detection mechanism is provided. FIG. 3 illustrates schematically a disc structure which provides various upstream sample processing steps, as described in the art, followed by injection of bead-based sandwich immunoassay concentration into a flow channel. As before, it is understood that other disc structures not shown are used for preparation of the sample and delivery to a mixing chamber which is shown. In the mixing chamber the beads, sample, and reagents are incubated and are present in solution at the beginning of a wash process. Two waste chambers are connected to the mixing chamber. Waste 1 has a volume approximately equal to the volume of solution to be applied to the mixing chamber and is connected to the mixing chamber via capillary valve V1. Waste 2 receives the bead-based sandwich immunoassay solution that is injected through the flow channel and is connected by a capillary valve V3.

As the disc rotates and the beads proceed through the flow channel, the fluorescence from the plurality of reporter labels within each bead sandwich is measured by a first optical detection channel. Simultaneously, the fluorescence from the reference labels within each bead is measured by a second optical detection channel. Upon division of the reporter label fluorescence measurement by the reference label fluorescence measurement. The normalised immunoassay signal is similarly calculated by Mathematical adjustment, for example by dividing the reporter measurement by the reference measurement, as described before.

The length of the flow channel and the flow rate of the bead-based solution are designed to ensure that no beads flow through the flow channel without being fluorescently measured.

Multiplexed assays may be accommodated through differential staining with well-separated emission peaks in concert with the reference label. It will be appreciated that magnets may also be used to drag beads to a single detection point.

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. CD ROM, or magnetic recording medium, e.g. a floppy disk or hard disk. The carrier may be an electrical or optical signal which may be transmitted via an electrical or an optical cable or by radio or other means.

In the specification the terms “comprise, comprises, comprised and comprising” or any variation thereof and the terms include, includes, included and including” or any variation thereof are considered to be totally interchangeable and they should all be afforded the widest possible interpretation and vice versa.

The invention is not limited to the embodiments hereinbefore described but may be varied in both construction and detail.

1. A microfluidic system for processing biological samples comprising:
a platform coupled to a rotary motor and adapted to provide at least one detection chamber for receiving particles, said particles comprising a reference label and reporter label, wherein the labels exhibit different wavelength properties when irradiated with light; and
means for normalising the particle reporter label in the detection chamber by using any detected variance in the measured properties of the reference label.

2. The microfluidic system as claimed in claim 1 wherein the means for normalising comprises determining the magnitude of the measured fluorescence of the reference label, wherein the determined magnitude provides a correction factor for the measured fluorescence of the reporter label.

3. The microfluidic system of claim 1 wherein the measured properties of the reference label comprises measured fluorescence properties of the reference label.

4. The microfluidic system as claimed in claim 3 wherein the measured fluorescence properties of the reference numeral provides a normalising factor for measured fluorescence of the reporter label.

5. The microfluidic system as claimed in claim 3 wherein the measured fluorescence properties of the reference numeral provides a normalising factor for measured fluorescence of the reporter label and the normalising factor is obtained by dividing the reporter label fluorescence measurement by the reference label fluorescence measurement, such that a normalised immunoassay signal will be corrected for any errors in the system.

6. The microfluidic system as claimed in claim 1 wherein the particles are immuno-modified beads and/or fluorescently labelled immuno-modified beads representative of characteristics of said biological sample.

7. The microfluidic system as claimed in claim 1 wherein the detection zone is adapted to cooperate with an optical system while the platform rotates.

8. The microfluidic system as claimed in claim 1 wherein the particle receiving structure comprises a pelleting chamber.

9. The microfluidic system as claimed in claim 1 wherein the particle receiving structure comprises a flow channel.

10. A method of processing biological samples in a microfluidic system comprising the steps of:
positioning at least one detection chamber adapted for receiving particles, said particles comprising a reference label and reporter label, wherein the labels exhibit different wavelength properties when irradiated with light; and
normalising the particle reporter label by using any detected variance in the measured properties of the reference label.

11. The method of claim 10 comprising the step measuring properties of the reference label by measuring fluorescence properties of the reference label.

12. The method of claim 11 wherein the measured fluorescence properties of the reference numeral provides a normalising factor for measured fluorescence of the reporter label.

13. The method of claim 10 comprising the step of obtaining the normalising factor by dividing the reporter label fluorescence measurement by a reference label fluorescence measurement, such that a normalised immunoassay signal will be corrected for any errors in the system.

14. The method of claim 10 wherein the particles are immuno-modified beads and/or fluorescently labelled immuno-modified beads representative of characteristics of said biological sample.

15. The method of claim 10 wherein the particle receiving structure comprises a pelleting chamber.

16. Use of a reference label from a particle to establish if an adequate number of particles are present in a detection chamber for a viable immunoassay measurement according to the method of claim 10.
